(19) United States
(12) Patent Application Publication
(10) Pub. No.: US 2025/0177732 A1
(43) Pub. Date: Jun. 5, 2025

(54) SKULL ANCHOR BOLT WITH A CONTROLLED BREAK POINT AND RECOVERY MEANS

(71) Applicant: Ad-Tech Medical Instrument Corporation, Oak Creek, WI (US)

(72) Inventors: Jacob Wagner, New Berlin, WI (US); Devon Moloney, Milwaukee, WI (US); Garrick Zinsli, Waukesha, WI (US)

(73) Assignee: Ad-Tech Medical Instrument Corporation, Oak Creek, WI (US)

(21) Appl. No.: 18/964,879

(22) Filed: Dec. 2, 2024

Related U.S. Application Data
(60) Provisional application No. 63/605,279, filed on Dec. 1, 2023.

(51) Int. Cl. A61N 1/05

(52) U.S. Cl. CPC A61N 1/0539

(57) ABSTRACT

An improvement to anchor bolts adapted to be screwed into a patient's skull for the purpose of placing a depth electrode at a desired location within the patient's cranium. The improved anchor bolt includes an engineered controlled break point and recovery means such as a secondary fitting to facilitate removal if the anchor bolt is impacted and breaks or bends at the controlled break point. The anchor bolt is intended to be used in a similar manner to existing anchor bolts with the added benefits of protecting the patient from excessive harm from violent impact of anchor bolts which can occur during seizures for example, as well as facilitating removal of remaining parts of a damaged anchor bolt without the need for surgical intervention and in a relatively easy fashion.

### CROSS-REFERENCE TO RELATED APPLICATION

The present application claims priority of U.S. Provisional Patent Application No. 63/605,279, filed Dec. 1, 2023, the content of which is incorporated herein by reference in its entirety.

### FIELD OF THE INVENTION

The present invention relates generally to anchor bolts used in the skull of a patient for subsequent placement of a depth electrode used in neurosurgery, for example, to monitor or treat epilepsy or other neurological disorders. More particularly, the invention relates to an anchor bolt with a controlled break point and recovery means.

### BACKGROUND OF THE INVENTION

Depth electrode anchor bolts are screwed into burr holes pre-drilled into the skull of a patient for subsequent placement of depth electrodes. An anchor bolt has an internal lumen for the depth electrode to permit access inside a patient's skull, threads on the outer surface of the distal end referred to as skull threads, and threads on the outer surface of the proximal end referred to as cap threads.

The proximal end typically has an axial socket or broach, such as a square broach, that is used to turn and drive the anchor bolt so that the skull threads are accurately secured in the patient's skull. Stereotactic equipment is often used to implant the anchor bolts and to later place depth electrodes through the lumen to the appropriate location in the patient's brain tissue.

The depth electrode and stylet are inserted through the lumen in the anchor bolt into the cranium. The stylet is removed after the distal end of the depth electrode is placed by the medical staff. Once the depth electrode is placed at the intended location, the cap is tightened to fix the location of the depth electrode and stabilize the depth electrode during intraoperative electroclinical monitoring. An internal gasket minimizes concerns about potential cerebrospinal fluid leakage and infection of the subdural space. The anchor bolt is designed to withstand longitudinal pulling and pushing forces and a certain amount of lateral force as well. The internal diameter of the lumen differs to match the type of depth electrode being used, and anchor bolts are also made to have different lengths.

The anchor bolts need to have appropriate strength in order to ensure reliable installation and removal, and also maintain the integrity of the placement of the distal end of the depth electrodes. Titanium Grade 2 is often used to make anchor bolts for depth electrodes. Titanium is a non-ferrous, biocompatible material that has been found conditionally suitable for use in magnetic resonance imaging. One issue with anchor bolts is that the proximal end is susceptible to being unintentionally knocked and laterally displaced, for example if the patient has a seizure. If the anchor bolts are made to withstand a substantial force, the skull threads can loosen when knocked and the distal end of the depth electrode can be displaced which is not desirable. Anchor bolts with a longer external stem are more susceptible to loosening when knocked. To avoid this issue, some manufacturers have made anchor bolts with thinner walls that bend or fracture if knocked with a substantial lateral force. One issue with these types of anchor bolts is that bent or broken anchor bolts can be difficult and time consuming to remove, especially when broken at or below the patient's skin. Sometimes surgical intervention is required to remove the remaining parts.

### SUMMARY OF THE INVENTION

The invention is an improvement to anchor bolts adapted to be screwed into a patient's skull for the purpose of placing a depth electrode at a desired location within the patient's cranium. The improved anchor bolt includes an engineered controlled break point and recovery means such as a secondary fitting to facilitate removal if the anchor bolt is impacted and breaks or bends at the controlled break point. The anchor bolt is intended to be used in a similar manner to existing anchor bolts with the added benefits of protecting the patient from excessive harm from violent impact of anchor bolts which can occur during seizures for example, as well as facilitating removal of remaining parts of a damaged anchor bolt without the need for surgical intervention and in a relatively easy fashion.

More particularly, an anchor bolt constructed in accordance with the invention has a threaded collar located at a proximal end of the anchor bolt, with cap threads on an outer surface of the collar. The anchor bolt also has a subdural stem located at the distal end of the anchor bolt, with skull threads on an outer surface of the subdural stem. An external stem is located between the collar and the subdural stem. There can be a transition section between the threaded collar and the subdural stem. The anchor bolt has an axial broach in the collar accessible from the proximal end of the anchor bolt which enables a driver wrench head or fitting to turn the anchor bolt clockwise or counterclockwise around the longitudinal axis. This is the primary means for placing the anchor bolt into the patient's skull and removing the anchor bolt in normal use.

A recovery fitting section is located between the external stem and the subdural stem, and its outer surface desirably consists of a wrench fitting such as a hex or square fitting. The internal lumen extends along a longitudinal axis through the anchor bolt including through the collar, the external stem, the recovery fitting section, and the subdural stem and permits access for the depth electrode (and a stylet) inside the patient's skull. As mentioned, the improved anchor bolt has a controlled break point in the external stem or between the external stem and the recovery fitting. The anchor bolt is intended to be screwed into a pre-drilled hole in the patient's skull such that the subdural stem is embedded in the patient's skull and the remaining parts of the anchor bolt, including the recovery fitting, remain external to the patient's skull and skin. The controlled break point breaks preferentially when a significant lateral force is applied to the collar or external stem of the installed anchor bolt. This controlled break is preferrable to exacerbating patient injury by using anchor bolts that will not bend or break and is also preferrable to allowing the anchor bolt to possibly bend or break in the subdural stem. With the invention, the recovery fitting remains external to the patient's skull yet adjacent and connected to the subdural stem and easily accessible with a wrench to remove from the patient. Further, excessive torque is isolated from the subdural stem, further protecting the patient, and also ensuring the straightness and integrity of the subdural stem and recovery fitting to facilitate removal without exacerbating further injury.

The improved anchor bolt, as mentioned, is intended to be operated like prior art anchor bolts in normal use. An assembly including the anchor bolt recited in claim 1 further includes a threaded cap with an axial opening that is adapted to be tightened onto the threaded collar of the anchor bolt with the depth electrode being placed through the axial opening of the threaded cap. An internal gasket is located within the cap for holding and sealing the depth electrode once it is placed. An elastomeric cap covers the threaded cap and the tail of the depth electrode when the anchor bolt and the depth electrode are installed and in use. Desirably, all of the components, including the anchor bolt, are made from a non-ferrous, biocompatible material that is conditionally suitable for magnetic resonance imaging.

In the exemplary embodiments, the improved anchor bolt is made of Titanium Grade 23 which has a higher tensile strength than prior art anchor bolts using Titanium Grade 2. The purpose of the additional strength is to ensure that the anchor bolt will not fail if impacted with lateral force except at the controlled break point. In the exemplary embodiments the controlled break point is constructed by tapering the outer diameter of the external stem narrower as the external stem approaches the recovery fitting, which in turn renders the wall around the internal lumen thinner at that point. In the exemplary embodiment, the controlled break point is designed such that the lateral force necessary to break the anchor bolt is sufficient to cause a mechanical moment in the range of about 12-18 pound-inches at the controlled break point. This range of torque has been selected in order to provide significant protection to the patient while not enabling the anchor to prematurely break during normal use.

### BRIEF DESCRIPTION OF THE DRAWINGS

FIG. 1 is a side elevational view of an anchor bolt constructed in accordance with the prior art.

FIG. 1A is a left-side, end view of the prior art anchor bolt shown in FIG. 1.

FIGS. 2A through 2C show a prior art anchor bolt (FIG. 2A) in combination with a cap gasket (FIG. 2B) and a silicone cap (FIG. 2C).

FIG. 3A is a schematic illustration showing the steps involved for inserting an anchor bolt into a patient's skull, as is known in the art.

FIG. 3B is a schematic illustration showing the steps involved in removing an anchor bolt as is known in the art, assuming that the anchor bolt is not bent or fractured during use.

FIG. 4 is a perspective view of an anchor bolt constructed in accordance with the invention to have a controlled break point and recovery means.

FIG. 5 is a longitudinal-sectional view of an anchor bolt constructed in accordance with the invention illustrating a broach for installing and removing the anchor bolt under normal conditions.

FIG. 5A is an end view of the anchor bolt in FIG. 5 depicting the geometry of the broach.

FIG. 6 is a photograph showing examples of anchor bolts constructed in accordance with the invention prior to and after breaking at the controlled break point.

FIG. 7 is a side elevational view of an anchor bolt constructed in accordance with one exemplary embodiment of the invention.

FIG. 7A is a detailed view of the area depicted by circular arrow A in FIG. 7 showing the geometry of the controlled break point.

FIG. 8 is a side elevational view of an anchor bolt constructed in accordance with one exemplary embodiment of the invention.

### DETAILED DESCRIPTION OF THE DRAWINGS

### Background Prior Art

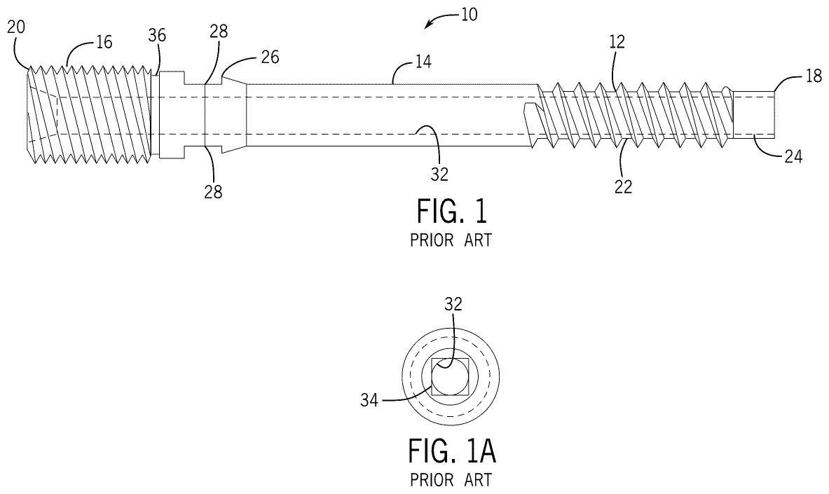

FIGS. 1 and 1A through FIGS. 3A and 3B describe an anchor bolt 10 constructed in accordance with the prior art and the installation and removal of the anchor bolt 10 as normally accomplished in the prior art. The anchor bolt 10 is adapted to be screwed into a patient's skull for the purpose of placing a depth electrode at a desired location within the patient's cranium. The anchor bolt 10 depicted in FIGS. 1 and 1A is a 26 mm anchor bolt 10. The anchor bolt 10 generally includes a subdural stem 12, an external stem 14 and a collar 16. The collar 16 is located at a proximal end 20 of the anchor bolt 10, and the subdural stem 12 is located at a distal end 18 of the anchor bolt 10. The subdural stem 12 includes threads 22 for screwing the anchor bolt 10 into burr holes pre-drilled into the skull of a patient. Regulations mandate that no more than 8 pound-inches of torque be required to screw the anchor bolt 10 into the skull of the patient. Using properly drilled burr holes, it is typical to take no more than 1½ to 2 pound-inches of torque to screw the anchor bolt into the skull of a patient. The subdural stem 12 also includes an unthreaded lead 24 which is useful for initially setting the anchor bolt in the burr hole. In the example of anchor bolt shown in FIG. 1 the subdural stem has a length of approximately 0.4 inches including the unthreaded lead portion 24.

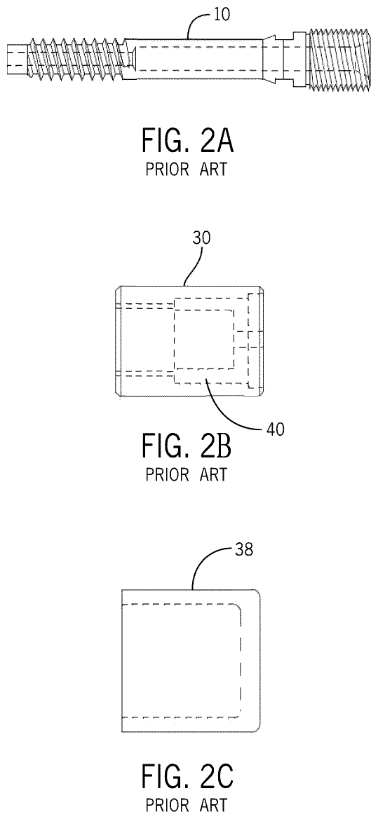

The external stem 14 extends between the subdural stem 12 and the collar portion 16. The external stem 14 in this example is about 0.625 inches in length, and the length of the collar 16 is approximately 0.40 inches. The collar 16 includes a threaded portion 16 for receiving a cap 30 (FIG. 2), as will be discussed in more detail in connection with FIGS. 2A through 2C. There is a transition portion 26 extending between the external stem 14 and the threaded portion of the collar 16. This transition portion 26 includes flat indentations 28. A cylindrical lumen 32 extends longitudinally through the anchor bolt 12 generally from the proximal end 20 to the distal end 18. The diameter of the lumen 32 in FIG. 1 is constant through the entire length of the anchor bolt 10 except at the mouth the proximal end 20 and in the collar where the lumen 32 is surrounded by a broach 34 used to turn the anchor bolt 10 for placement and removal. In some anchor bolts, the diameter of the lumen in the subdural stem is smaller than the diameter of the lumen in the external stem. There is also circumferential relief 36 between the transition portion 26 and the threaded collar portion 16. Referring to FIG. 1A, the broach 34 in this example has a square shape and provides a socket for a driving wrench with a square head or fitting. The anchor bolt 10 depicted in FIG. 1, as mentioned previously, is often made from titanium grade 2 which is a non-ferrous biocompatible material that is conditionally suitable for magnetic resonance imaging.

Referring to FIGS. 2A through 2C, the anchor bolt 10 a collar 30 and a silicone cap 38 are shown. The anchor bolt cap 30 is also desirably made of titanium grade 2 or another non-ferrous biocompatible material that is conditionally suitable for magnetic resonance imaging. The anchor bolt cap 30 contains an internal gasket 40 made from an elastomeric silicone product. The internal gasket 40 has an opening through the top of the cap through which the depth electrode passes when it is installed.

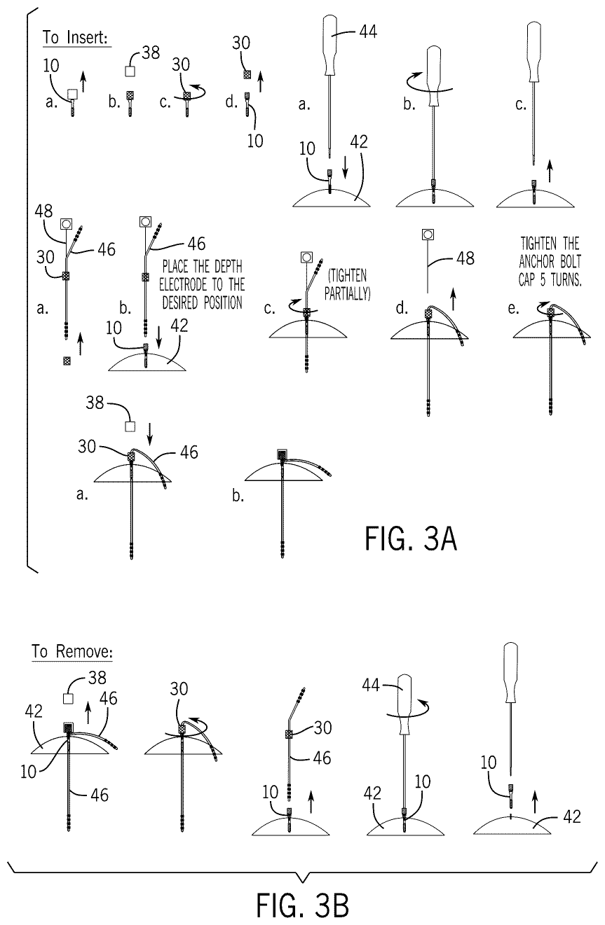

Turning now to FIG. 3A, the steps for inserting the anchor bolt 10 and a depth electrode 46 are described in connection with the illustrations in FIG. 3A. Referring to step 1A, the anchor bolt 10 is provided with the titanium threaded cap 30 and the silicone cap 38 in place on the anchor bolt 10. In step 1B, the silicone cap 38 is removed and, in step 1C, the titanium threaded cap is removed from the anchor bolt 10 as depicted in step 1D. In step 2A, the anchor bolt 10 is placed in a burr hole in the patient's skull 42 and a driver wrench 44 having a head that fits into the broach 34 on the proximal end 20 of the anchor bolt 10 is engaged. Step 1B shows the driver wrench 44 being turned to screw the threads 22 on the subdural stem 12 into the skull 42. When the anchor bolt 10 has been secured to a suitable depth, the driver wrench 44 is disengaged, see step 2C. In step 3A, the depth electrode 46 along with a stylet 48 are slid through the titanium threaded cap 30 and through the internal silicone gasket 40 located within the titanium threaded cap 30. As shown in the illustration for step 3A, the titanium threaded cap 30 is passed over the distal ends of the stylet 48 and the depth electrode 46. In step 3B the depth electrode 46 is placed to the desired position through the anchor bolt 10. In step 3C, the threaded cap 30 is tightened partially to hold the depth electrode 46 in place. The stylet 48 is then removed, see step 3D and the threaded cap 30 is tightened to seal the internal gasket 40 against the depth electrode 46. In step 4B, the silicone cap 38 is placed over the threaded titanium cap 30 with the depth electrode 46 being fed under the silicone cap 38 and made available for connection to EEG (electroencephalogram) equipment or to other neuromonitoring equipment for analyzing stimulated brain activity.

FIG. 3B shows the steps for removing the depth electrode 46 and the anchor bolt 10 under normal conditions. Step 1 shows the depth electrode 46 and the anchor bolt placed in the skull 42 of a patient with the silicone cap 38 placed over the threaded cap 30 on the anchor bolt 10. Step 1 further shows the silicone cap 38 being removed. Step 2 in FIG. 3B is to loosen the threaded cap 30 from the external threads on anchor bolt 10. Step 3 in FIG. 3B illustrates the removal of the depth electrode 46 and the threaded titanium cap 30. Step 4 in FIG. 3B shows the driver wrench 44 being used to unscrew the anchor bolt 10 from the skull 42 of the patient. Step 5 in FIG. 3B shows the anchor bolt 10 fully removed from the skull of the patient.

As illustrated in FIG. 3B, removal of depth electrodes 46 and the anchor bolts 10 under normal circumstances is relatively straight forward. Unfortunately, knocking anchor bolt 10 can cause it to break or bend which makes the removal process more difficult, sometimes requiring surgical intervention to remove the retained portion of the anchor bolt.

### Description of the Invention

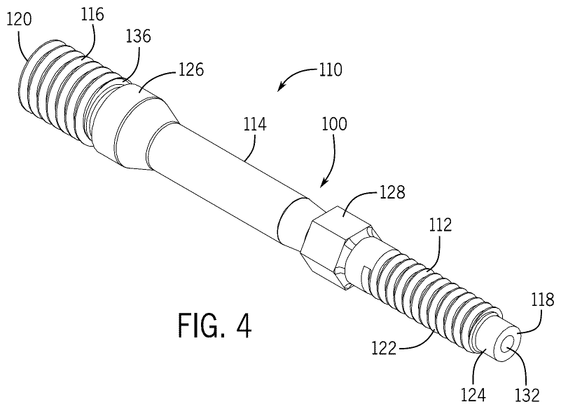

FIG. 4 is a perspective view of an anchor bolt 110 having a controlled break point 100 and a recovery fitting 128 in accordance with the invention. The anchor bolt 110 is desirably made of a titanium alloy, or another non-ferrous bio-compatible material, having a stronger tensile strength than the titanium T2 used in connection with the anchor bolt 10 described in FIGS. 1 and 2. The exemplary anchor bolt 110 illustrated in FIG. 4 is a 26 mm anchor bolt similar to the anchor bolt described in FIGS. 1 and 2, however, it should be understood that the invention is suitable for use with 21 mm anchor bolts or anchor bolts having lengths. By using a material that has a higher tensile strength, the anchor bolt 110 is less susceptible to breakage or bending than the prior art anchor bolts 10 described in connection with FIGS. 1 and 2, except for at the controlled break point 100 in the anchor bolt 110. Using the stronger material means that it is less likely for the subdural stem 112 in the anchor bolt 110 to bend or break than the subdural stem 12 in a prior art anchor bolt 10. It is also less likely for the external stem 114 in the anchor bolt 110 to bend or break than the external stem 14 in a prior art anchor bolt 10. The strength of the material of the anchor bolt 110 and the design of the controlled break point 100 are selected in combination, preferably, so that the anchor bolt 110 will break at the controlled breakpoint 100 when a lateral force is applied to the cap threads 116 or external stem 114 sufficient to create torque in the range of 12 to 18 pound-inches at the controlled break point 100.

The anchor bolt 110 also includes a recovery fitting 128 between the subdural stem 112 and the break point 100 at the base of the external stem 114. The recovery fitting 128 is provided to facilitate removal of the subdural stem 112 from the patient's skull in the event that the anchor bolt 110 fails at the controlled break point 100. The recovery fitting 128 is preferably a hex fitting to facilitate removal with a common wrench fitted over the recovery fitting 128 transversely to the longitudinal axis of the anchor bolt 110, or the remaining subdural stem 112B. Alternatively, an axial socket wrench could be used to engage the recovery fitting 128 and remove remaining portions of the subdural stem 112, if failure occurs. Other types of fittings such as a square fitting or other shapes can be used to carry out the invention. In other aspects, the anchor bolt 110 is intended to be similar to and compatible with the use of the prior art anchor bolts 10 described for example in FIGS. 1-3.

Still referring to FIG. 4, the anchor bolt 110 has a subdural stem 112 at its distal end 118, which includes an unthreaded lead portion 124 and subdural threads 122. The anchor bolt 110 in FIG. 4 also shows the opening of an internal lumen 34 at its distal end 118. The anchor bolt 110 also includes an external stem 114 and a threaded collar 116 with the transition portion 126 and a circumferential relief 136 located between the external stem 114 and the threaded collar 116. In accordance with the invention, the controlled break point 100 is located as part of the external stem 114 and desirably at the interface between the external stem 114 and the recovery fitting 128. The recovery fitting 128 is located adjacent the subdural stem 112 such that it can be used to remove the subdural stem 112 from a patient's skull 42 in the event that the anchor bolt 110 bends or breaks at the controlled break point 100. It is intended that the anchor bolt 110 be placed in the patient's skull to a depth leaving the recovery fitting 128 extracorporeally accessible so that it is easily accessible in the event the anchor bolt 110 breaks.

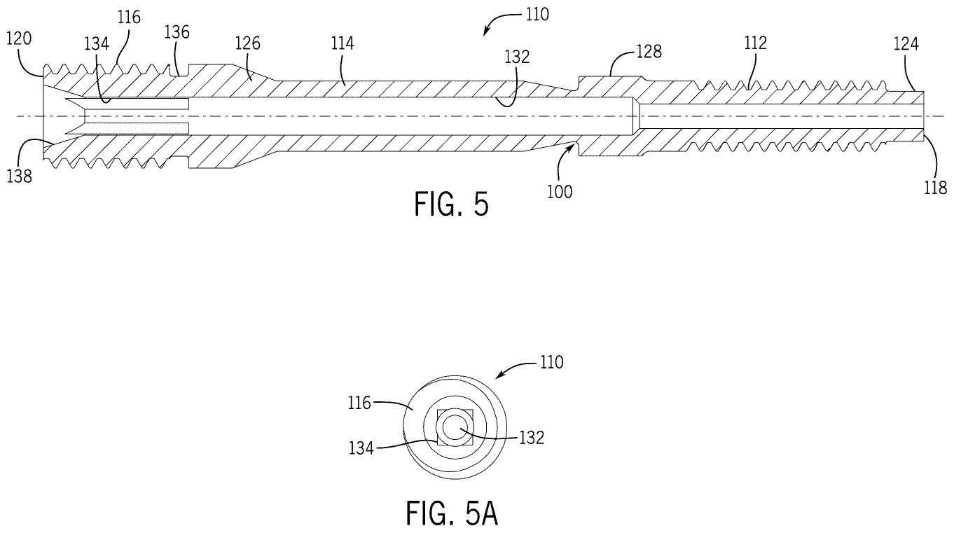

FIG. 5 shows a longitudinal section view of an anchor bolt 110 constructed in accordance with the invention which illustrates the broach 134 on the proximal end 120 of the anchor bolt 110. FIG. 5A shows an end view of the anchor bolt 110 from the proximal side 120 of the anchor bolt 100. The broach 134 shown is a square socket that extends into the collar 116, like in the prior art, and is adapted to receive a driver wrench with a square head or fitting as described in connection with the prior anchor bolts 10. The shape of the broach 134 is not limited to being a square and can take on other shapes within the spirit of the invention. It is intended that the anchor bolt 110 be placed and removed under normal conditions in accordance with the steps described in connection with FIG. 3. However, as mentioned, in the event that the anchor bolt 110 is knocked in use and bent or broken, the anchor bolt 110 is designed to fail at the controlled break point 100 with the recovery fitting 128 being used to remove the anchor bolt 110 from the patient's skull.

The threaded collar 116 also includes a tapered mouth 138 providing an opening from the proximal end 120 of the anchor bolt 110 into the internal lumen 132 and into the broach or socket 134. The tapering of the mouth 138 helps locating the head of the driver wrench into the broach 134, and also feeding the stylet 48 and the depth electrode 46 into the lumen 132. The embodiment shown in FIGS. 5 and 5A has a lumen 132 with two stages having different diameters with smaller diameter in the subdural stem 112.

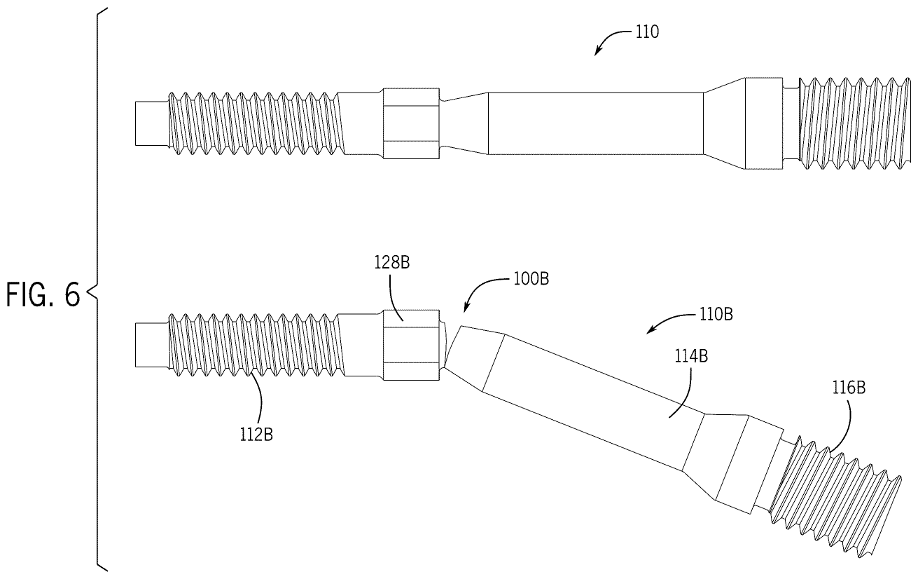

FIG. 6 is a photograph illustrating an anchor bolt 110 constructed in accordance with the invention in its normal state and also an anchor bolt 110B that has received a lateral force in testing against threaded collar 116B which has caused the anchor bolt 110B to fail at the controlled break point 100B. The testing was accomplished by securing the anchor bolt 110B in its unbroken state into a substrate, namely 40 PCF bone analog polyurethane foam material made for biomechanical testing, and applying a measured amount of lateral force against the threaded collar 116 in its unbroken state. It is desirable that the anchor bolt 110 be able to consistently withstand a lateral force up to a threshold, e.g. 12 to 18 pound-inches, but also reliably fail at the controlled break point 100 when the threshold amount to lateral force occurs in order to protect the patient, e.g. in case that the anchor bolts 110 are violently impacted during an epileptic seizure or otherwise. As can be seen in FIG. 6, the subdural stem 112B and the recovery fitting 128B remain intact and generally undamaged even though the anchor bolt 110B mechanically failed at the controlled break point 100B. Accordingly, even if the anchor bolt 110B is broken as shown in FIG. 6, the user can easily use a wrench to remove the subdural stem 112B from the patient in a relatively straightforward manner without causing further damage to the patients skull or skin.

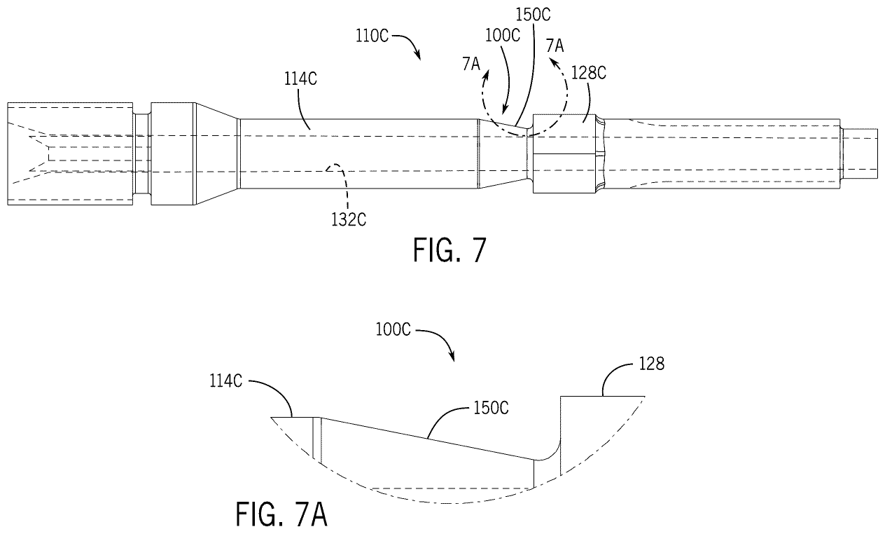

FIGS. 7 and 7A illustrate an anchor bolt 110C manufactured and constructed in accordance with one embodiment of the invention. The anchor bolt 110C in FIG. 7 is made of titanium grade 23 which has a higher tensile strength than titanium grade 2 used in the prior art anchor bolts described in FIG. 1. The anchor bolt 110C in FIG. 7 is a 26 mm anchor bolt and has a lumen 132C with a constant internal diameter of 0.49 inches within tolerance. The outer diameter of the external stem 114C in this embodiment is 0.100 inches within tolerance, which means that the wall thickness in the main part of external stem 114C is approximately 0.025 inches within tolerance. The controlled break point 100C is configured by tapering the outer wall of the external stem 114C to become narrower as it approaches the recovery fitting 128C, see tapered outer wall portion 150C. The minimum diameter of the tapered wall section 150C, which is adjacent to the recovery fitting 128C, in this example is 0.79 inches within tolerance which means that the wall thickness at the minimum diameter is 0.015 inches within intolerance. The length of the tapered portion 150C forming the controlled break point 100C in this embodiment is approximately 0.09 inches within tolerance. Applicant has found that the following configuration of the controlled break point 100C, if titanium grade 23 is used as a material, reliably causes mechanical failure to occur at the controlled break point 100C when a lateral force is applied to the collar 116 to cause a moment in the range of 12 to 18 pound-inches at the controlled break point 100C.

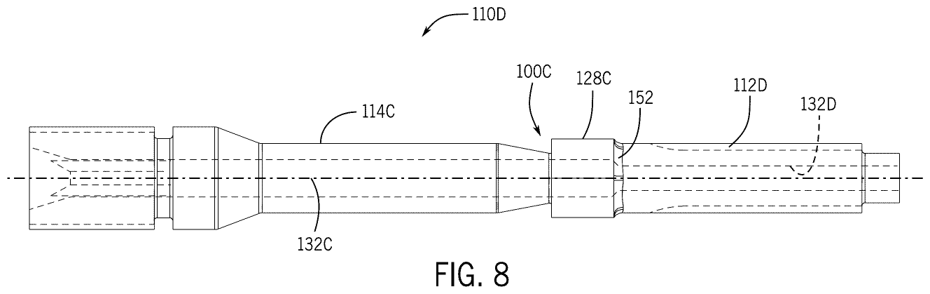

FIG. 8 illustrates another anchor bolt 110D manufactured and constructed in accordance with the invention. The anchor bolt 110D in FIG. 8 is made of titanium grade 23, like the anchor bolt 110C in FIG. 7, and it is also a 26 mm anchor bolt. The primary difference in anchor bolt 110D is that that internal lumen 132C, 132D has a narrower diameter in the subdural stem 112D. The diameter of the lumen in the subdural stem is 0.39 inches within tolerance. The diameter of the lumen 132C in the external stem 114C is 0.49 inches within tolerance which is the same as the diameter of lumen 132C in the anchor bolt 110C in FIG. 7. In FIG. 8, there is a transition 152 in the subdural stem 112 D adjacent the recovery fitting 128C which reduces the diameter of the internal lumen. The dimensions of the anchor bolt 110D in FIG. 8 are otherwise the same as the dimensions of the anchor bolt 110C in FIG. 7. The configuration and the dimensions of the controlled break point 100C, including the nature of the tapered portion 150C, are also the same since the material of the anchor bolts is the same and the dimensions of the external stem 114C, the recovery fitting 128C and the internal lumen portion 132C extending through the external stem 114C and recovery fitting 128C are the same.

The invention has been described in connection with various exemplary embodiments. The scope of the invention is not intended to be limited by the materials and or specific dimensions described in these exemplary embodiments.

We claim:

1. An anchor bolt adapted to be screwed into a patient's skull for the purpose of placing a depth electrode at a desired location within the patient's cranium, the anchor bolt comprising:
a collar located at a proximal end of the anchor bolt;
cap threads on an outer surface of the collar;
a subdural stem located at a distal end of the anchor bolt;
skull threads on an outer surface of the subdural stem;
an external stem;
a recovery fitting section located between the external stem and the subdural stem;
an internal lumen extending along a longitudinal axis through the anchor bolt including through the collar, the external stem, the recovery fitting section, and the subdural stem;
a controlled break point in the external stem or between the external stem and the recovery fitting;
an axial broach in the collar accessible from the proximal end of the anchor bolt, said broach adapted to enable a wrench or fitting to turn the anchor bolt clockwise or counterclockwise around the longitudinal axis;
wherein said anchor bolt is adapted to be screwed into a pre-drilled hole in the patient's skull such that the subdural stem is embedded in the patient's skull and the remaining parts of the anchor bolt remain external to the patient's skull and skin with the recovery fitting section being closer to the subdural stem than the controlled break point, and the internal lumen permitting access for a depth electrode inside the patient's skull;
and further wherein the controlled break point comprises a portion of the anchor bolt that breaks preferentially when a lateral force is applied to the collar or external stem of the installed anchor bolt.

2. An assembly including the anchor bolt recited in claim 1 further comprising:
a. a threaded cap with an axial opening that is adapted to be tighten onto the threaded collar of the anchor bolt with the depth electrode being placed through the axial opening of the threaded cap; and
b. an internal gasket located within the cap and also having an opening through which the depth electrode is placed.

3. The assembly recited in claim 2 further comprising an elastomeric cap that covers the threaded cap and the tail of the depth electrode when the anchor bolt and the depth electrode are installed and in use.

4. The assembly recited in claim 2 wherein the components are each made from a non-ferrous, biocompatible material that is conditionally suitable for magnetic resonance imaging.

5. The anchor bolt recited in claim 1 made of Titanium Grade 23.

6. The anchor bolt recited in claim 1 wherein the controlled break point is constructed by tapering the outer diameter of the external stem narrower as the external stem approaches the recovery fitting.

7. The anchor bolt recited in claim 1 wherein the lateral force necessary to break the anchor bolt is sufficient to cause a moment in the range of 12-18 pound-inches at the controlled break point.

8. The anchor bolt recited in claim 1 wherein the internal lumen has a constant diameter within tolerance through the external stem, the recovery fitting section, and the subdural stem.

9. The anchor bolt recited in claim 1 wherein the internal lumen in the external stem has a first diameter, and the internal lumen in the subdural stem has a second diameter that is smaller than the diameter of the internal lumen in the external stem.
